(19) United States
(12) Patent Application Publication
(10) Pub. No.: US 2009/0196917 A1
(43) Pub. Date: Aug. 6, 2009

(54) Liposomal Formulations of Hydrophobic Lactone Drugs in the Presence of Metal Ions

(75) Inventors: Vijay Joguparthi, Lexington, KY (US); Bradley D. Anderson, Lexington, KY (US)

Correspondence Address: CROWELL & MORING LLP;INTELLECTUAL PROPERTY GROUP, P.O. BOX 14300, WASHINGTON, DC, 20044-4300, US

(73) Assignee: University of Kentucky Research Foundation, Lexington, KY (US)

(21) Appl. No.: 12/363,394

(22) Filed: Jan. 30, 2009

Related U.S. Application Data
(60) Provisional application No. 61/025,601, filed on Feb. 1, 2008.

(51) Int. Cl. A61K 9/127; A61K 31/4745; A61K 31/35

(52) U.S. Cl. 424/450; 514/283; 514/460

(57) ABSTRACT

Disclosed herein is a liposomal formulation that comprises a liposome and a liquid carrier. In the liposomal formulation, the liposome comprises a hydrophobic lactone drug and has an intraliposomal metal ion concentration higher than the metal ion concentration of the liquid carrier. Also disclosed herein is a method for active loading of a hydrophobic lactone drug into a liposome. The method includes preparing a liposome in the presence of a metal salt solution, an acid form of a counterion of the metal salt being membrane permeable, such that the liposome preparation contains entrapped metal ion. The method further includes forming a liposome with high intraliposomal pH by separating extravesicular metal salt solution from the liposome by exposing the liposome to a metal salt-free solution, resulting in diffusion of the acid form of the counterion out of the liposome and formation of an intraliposomal pH higher than that of the metal salt-free solution. The method also includes exposing the liposome with high intraliposomal pH to an isoosmolal solution containing a hydrophobic lactone drug, the isoosmolal solution having a pH lower than the intraliposomal pH, such that the hydrophobic lactone drug accumulates in the liposome predominantly in its ring-opened form.

### CLAIM OF PRIORITY

This application claims priority under 35 U.S.C. § 119 to U.S. Provisional Application No. 61/025,601, filed Feb. 1, 2008, the entire contents of which are hereby incorporated by reference.

### STATEMENTS REGARDING FEDERALLY SPONSORED RESEARCH

The invention was funded in part by Grant Nos. R01CA87061 awarded by the National Institutes of Health and National Cancer Institute. The government has certain rights in the invention.

### FIELD OF ART

The liposomal formulations, their associated methods of making, and their associated methods of use disclosed herein relate to delivering pharmaceutically effective amounts of hydrophobic lactone drugs.

### BACKGROUND

Liposomes are spherical nanoparticles comprising one or more concentric lipid bilayers enclosing an aqueous interior. Liposomes with a single concentric bilayer have a typical size range of ˜50-200 nm and are referred to as unilamellar vesicles. Liposomes with more than one concentric bilayer are referred to as multilamellar vesicles.

Liposomes can release drugs to a target tissue or can release drugs in circulation. Unilamellar vesicles are of particular pharmaceutical use in targeting specific tissues in the body, such as the spleen or tumors.

Liposomes can be used to encapsulate both hydrophilic and hydrophobic drug molecules. Hydrophobic molecules are thought to partition into the lipid bilayer, thereby gaining protection against a variety of reactions which they are prone to in the aqueous phase. However, liposomes can also be used as carriers for hydrophilic molecules by entrapping these molecules in the aqueous core of liposomes.

Liposomes are particularly suited to the delivery of chemotherapeutic drugs to tumors. Encapsulation of chemotherapeutics in liposomes is advantageous because liposomes preferentially accumulate in tumors and can avoid exposing healthy tissue to the chemotherapeutics avoiding undesirable side effects. However, in order to target specific tissue, the liposomes must retain the entrapped drug while in circulation to allow sufficient time for accumulation of the liposomes in the target tissue. Upon accumulation in the target tissue, the liposomes must then release the entrapped drug.

A prominent class of chemotherapeutic drugs are camptothecins. Camptothecins fall within the larger class of hydrophobic lactone drugs.

There is a need for novel formulation techniques for improved liposomal loading, improved liposomal retention, and prolonged liposomal release of camptothecins and similar hydrophobic lactone drugs.

### SUMMARY

Disclosed herein is a liposomal formulation comprising a liposome and a liquid carrier, the liposome comprising a hydrophobic lactone drug and having an intraliposomal metal ion concentration higher than the metal ion concentration of the liquid carrier.

Also disclosed herein is a method for active loading of a hydrophobic lactone drug into a liposome, the method comprising: preparing a liposome in the presence of a metal salt solution, an acid form of a counterion of the metal salt being membrane permeable, such that the liposome preparation contains entrapped metal ion; forming a liposome with high intraliposomal pH by separating extravesicular metal salt solution from the liposome by exposing the liposome to a metal salt-free solution, resulting in diffusion of the acid form of the counterion out of the liposome and formation of an intraliposomal pH higher than that of the metal salt-free solution; and exposing the liposome with high intraliposomal pH to an isoosmolal solution containing a hydrophobic lactone drug, the isoosmolal solution having a pH lower than the intraliposomal pH, such that the hydrophobic lactone drug accumulates in the liposome predominantly in its ring-opened form.

Among other factors, the liposomal formulation can exhibit increased liposomal loading, improved liposomal retention, and prolonged liposomal release of the hydrophobic lactone drug. Similarly, the active loading method can increase liposomal loading, improve liposomal retention, and prolong liposomal release of the hydrophobic lactone drug.

Other methods, features and advantages of the present invention will be or become apparent to one with skill in the art upon examination of the following detailed descriptions. It is intended that all such additional methods, features and advantages be included within this description, be within the scope of the present invention, and be protected by the accompanying claims.

### BRIEF DESCRIPTION OF THE DRAWINGS

FIG. 1 illustrates active loading of DB-67 into liposomes.

FIG. 2 illustrates a comparison of concentration of DB-67 achieved by active versus passive loading techniques.

FIG. 3 illustrates release of DB-67 from liposomes prepared by active loading versus passive loading techniques.

FIG. 4 illustrates the concentration of intravesicular DB-67 over time during active loading.

FIG. 5 illustrates release of DB-67 from liposomes prepared by active loading at various lipid concentrations.

FIG. 6 illustrates in vivo release of DB-67 from blank vesicles spiked with DB-67, liposomes prepared by passive loading, and liposomes prepared by active loading.

FIG. 7 illustrates tumor volume as a function of time during treatment of non-small cell lung cancer (H460) in mice with various dosages of nonliposomal DB-67.

FIG. 8 illustrates a dosing schedule and survival fraction during treatment of non-small cell lung cancer (H460) in mice with various dosages of nonliposomal DB-67.

### DETAILED DESCRIPTION

Before the present compositions and methods are described, it is to be understood that the invention is not limited to the particular methodologies, protocols, assays, and reagents described, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a,” “an,” and “the” include plural references unless the context clearly dictates otherwise.

Unless defined otherwise, all technical and scientific terms used herein have the same meanings as commonly understood by one of ordinary skill in the art to which this invention belongs. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

As used herein, the term “pharmaceutically effective amount” refers to an amount of an agent, reagent, compound, composition, or combination of reagents disclosed herein that, when administered to a subject, is sufficient to be effective against the disease state, including cancer.

The term “cancer” embraces a collection of malignancies with each cancer of each organ consisting of numerous subsets. Typically, at the time of cancer diagnosis, cancer consists in fact of multiple subpopulations of cells with diverse genetic, biochemical, immunologic, and biologic characteristics. Cancers may include, but are not limited to melanomas (e.g., cutaneous melanoma, metastatic melanomas, and intraocular melanomas), prostate cancer, lymphomas (e.g., cutaneous T-cell lymphoma, mycosis fungicides, Hodgkin's and non-Hodgkin's lymphomas, and primary central nervous system lymphomas), leukemias (e.g., pre-B cell acute lymphoblastic leukemia, chronic and acute lymphocytic leukemia, chronic and acute myelogenous leukemia, adult acute lymphoblastic leukemia, mature B-cell acute lymphoblastic leukemia, prolymphocytic leukemia, hairy cell leukemia, and T-cell chronic lymphocytic leukemia), and metastatic tumor. Cancer may be a solid tumor or a liquid tumor.

### Active Loading Method

Disclosed herein is an active method for loading a hydrophobic lactone drug into a liposome. The active loading method can increase loading and retention of the hydrophobic lactone drug in the liposome, and can prolong release of the hydrophobic lactone drug from the liposome.

Hydrophobic lactone drugs are present in different forms depending upon the pH of their environment. Hydrophobic lactone drugs contain a labile lactone moiety, which can undergo pH dependent reversible hydrolysis. Thus, the term “hydrophobic lactone drug” as used herein refers to a compound containing a labile lactone moiety that can be present in a form where the lactone ring is closed at low pH or a form where the lactone ring is open at high pH.

Camptothecins are exemplary hydrophobic lactone drugs and are a prominent class of chemotherapeutic agents that are cell cycle S-phase specific. The anti-cancer activity of camptothecins is primarily attributed to the intact lactone (Hertzberg et al., J. Med. Chem., 32 (3): 715-720, 1989). In aqueous solution, camptothecins undergo a pH dependent lactone ring hydrolysis to form inactive carboxylate species (Fassberg et al., J. Pharm. Sci., 81 (7): 676-684, 1992). Typically, for camptothecins, the lactone ring-opened form will be inactive and the lactone ring-closed form will be an active form of the drug, although this is not required (i.e. both forms may be active or at least exhibit some activity).

Camptothecins are typically used to treat cancer, including malignant solid tumors. DB-67 is a camptothecin that has displayed excellent anti-cancer activity in cell culture and small animal studies (Bom et al., J. Med. Chem., 42 (16): 3018-3022, 1999; Bom et al., J. Med. Chem., 43 (21): 3970-3980, 2000; Pollack et al., Cancer Res., 59: 4898-4905, 1999) and is currently in Phase I clinical studies at the University of Kentucky Markey Cancer Center. While human data are not yet available for DB-67, animal data indicate that its lactone to total AUC after intravenous administration is >90%. As a result of this outstanding stability, DB-67 may have pharmacologic and pharmacokinetic advantages over the currently approved camptothecins and many currently in development. DB-67, along with gimatecan and karenitecin, represents a new generation of camptothecin analogs that exhibit good blood stability and enhanced lipophilicity and potency.

Exemplary camptothecins include, but are not limited to, camptothecin, silatecan 7-t-butyldimethylsilyl-10-hydroxycamptothecin (DB-67), 7-ethyl-10-hydroxy-20(S)-camptothecin (SN-38), topotecan, irinotecan, 9-nitro-camptothecin, lurtotecan, exatecan, gimatecan, and karenitecin.

DB-67 is an exemplary camptothecin. The forms of DB-67 illustrated below demonstrate the pH dependent reversible hydrolysis of hydrophobic lactone drugs that transforms the drugs from their lactone ring-closed form to their lactone ring-opened form.

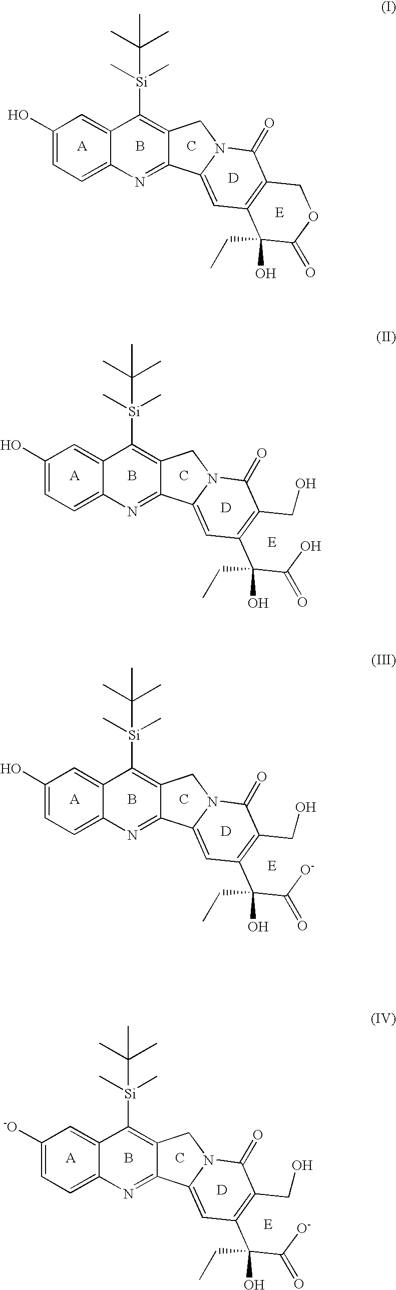

As shown above, DB-67, like other camptothecins, is subject to a pH dependent reversible hydrolysis of the α-hydroxy δ-lactone ring (E-ring) moiety to form DB-76 carboxylate anions. As a result, four different species of DB-67 exist depending upon pH. These four species are DB-67 lactone (I), DB-67 carboxylic acid (II), DB-67 carboxylate monoanion (III) and dianion (IV). DB-67 lactone (I) is the lactone ring-closed form of the drug and is membrane permeable.

Certain compounds of the camptothecin class possess an ionizable amine. These compounds are referred to herein as “ionizable amine-containing camptothecins.” Ionizable amine-containing camptothecins exist predominantly as cationic species at low pH (e.g. pH 2-7). Exemplary ionizable amine-containing camptothecins include lurotecan, topotecan, and irinotecan.

Other exemplary hydrophobic lactone drugs include, but are not limited to, statins, parthenolides, candimine, himbacine, narcotine, hydrastine, and homolycorine. It is well known in the art that the lactone moiety of statins, parthenolides, candimine, himbacine, narcotine, hydrastine, and homolycorine can undergo reversible hydrolysis.

Liposomal delivery is currently being investigated for various camptothecin analogues and several of these formulations are currently in preclinical or clinical trials (Emerson et al., Clin. Cancer Res., 6 (7): 2903-2912, 2000; Colbern et al., Clin. Cancer Res., 4 (12): 3077-3082, 1998; Tardi et al., Cancer Res., 60 (13): 3389-3393, 2000; Messerer et al., Clin. Cancer Res., 10 (19): 6638-6649, 2004; Pal et al., Anticancer Res., 25 (1A): 331-341, 2005; Seiden et al., Gynecol. Oncol., 93 (1): 229-232, 2004).

The challenge of delivering camptothecins and similar hydrophobic lactone drugs in liposomes lies in loading them in the liposomes, retaining them in the liposomes, and prolonging their release from the liposomes. Loading at therapeutic concentrations is complicated by their poor aqueous solubility. Prolonged retention is desired for liposome accumulation in the target tissue prior to drug release. Prolonged release is desired for less frequent drug dosing.

Efficient liposomal loading of camptothecins and similar hydrophobic lactone drugs is compromised by their poor aqueous solubility. To prepare liposomes by conventional methods such as hydration-extrusion or sonication, the drug must first be dissolved in an aqueous buffer. Alternatively, the drug is mixed with the lipid of interest in a suitable organic solvent and the solvent is evaporated to make a drug-lipid film. Then the film is hydrated with a polar solvent, such as water, to make liposomes. Thus, regardless of the method of preparation, an aqueous buffer has to be added at some stage of the formulation to make liposomes. Accordingly, liposomal loading of camptothecins and similar hydrophobic lactone drugs at therapeutically required concentration is thus impeded by their poor aqueous solubility.

In addition to loading challenges due to poor solubility, camptothecins and similar hydrophobic lactone drugs are poorly retained in liposomes. Prolonged drug retention in liposomes is often desired for tissue specific drug targeting. For example, in the case of cancer chemotherapy, a prolonged retention in liposomes is desired to allow enough time for liposomes to accumulate in tumor tissue. In such case, premature leakage of the encapsulated drug results in exposure of the healthy tissue to the drug, causing undesirable side effects.

Liposomes would appear to be ideal delivery systems for camptothecins and similar hydrophobic lactone drugs, especially if their release from the liposomes could be prolonged. Being relatively small and relatively lipophilic molecules, camptothecins exhibit large volumes of distribution and a narrow therapeutic index due to their accessibility and toxicity to normal tissues. However, long-circulating pegylated liposomes may reduce camptothecin distribution and toxicity in normal tissue. Long-circulating liposomes offer the possibility of prolonged drug release with less frequent dosing. Several studies have demonstrated the advantages of protracted camptothecin therapy (i.e., infusion regimens or multiple dosing over relatively frequent time intervals), but frequent dosing schedules are inconvenient to the patient and increase the cost of therapy. If camptothecin release from liposomes could be adequately prolonged, their activity could be extended allowing lower overall doses. Prolonged release needs to be addressed for similar hydrophobic lactone drugs as well.

The potential of using a high intraliposomal pH to maintain DB-67 in its membrane impermeable carboxylate monoanion (III) form was recently explored in order to develop prolonged release liposomal suspensions. V. Joguparthi, and B. D. Anderson. Liposomal delivery of hydrophobic weak acids: enhancement of drug retention using a high intraliposomal pH. J. Pharm. Sci. 97:433-454 (2008) and V. Joguparthi, T. X. Xiang, and B. D. Anderson. Liposome transport of hydrophobic drugs: gel phase lipid bilayer permeability and partitioning of the lactone form of a hydrophobic camptothecin, DB-76. J. Pharm. Sci. 97:400-420 (2008). However, a high intraliposomal pH could not be maintained under physiological conditions due to the rapid dissipation of the trans-membrane pH gradient by carbonate buffer (CO₂/H₂CO₃). V. Joguparthi, and B. D. Anderson. Liposomal delivery of hydrophobic weak acids: enhancement of drug retention using a high intraliposomal pH. J. Pharm. Sci. 97:433-454 (2008) and V. Joguparthi, S. Feng, and B. D. Anderson. Determination of intraliposomal pH and its effect on membrane partitioning and passive loading of a hydrophobic camptothecin, DB-67. Int. J. Pharm. 352:17-28 (2008). This inability to maintain a high intravesicular pH stimulated a search for alternative strategies to improve the retention of DB-67 and other similar hydrophobic lactone drugs.

Using high intraliposomal pH to load and retain a hydrophobic lactone drug is a form of passive drug loading. Passive drug loading refers to any technique for introducing drug to the liposomes directly during the process of making the liposomes. It involves passive diffusion of a drug from a drug solution exterior to the liposome into the interior aqueous compartment of the liposome. With passive diffusion, there is nothing inside the liposome to exchange for the entering drug. Passive drug loading is a well known method. However, it does not sufficiently concentrate the hydrophobic lactone drug inside the liposome due to the low aqueous solubility of the hydrophobic lactone drug.

In contrast, active drug loading refers to any technique for introducing drug into liposomes after the liposomes are prepared. It involves introducing a component into the interior aqueous compartment of the liposome that will exchange for the entering drug. Such exchange ultimately leads to increased concentration of the drug inside the liposome. In some cases, a combination of passive and active loading can be used to increase drug concentration inside the liposome.

The present method involves active loading of a hydrophobic lactone drug into a liposome in the presence of a metal ion gradient across the liposome and a pH gradient across the liposome.

The present method prepares a liposome in the presence of a metal salt solution. Such preparation entraps metal salt solution in the liposome. The metal salt solution contains a metal ion and a counterion of the metal salt. An acid form of the counterion of the metal salt is membrane permeable.

For example, the present method can prepare a liposome in the presence of a calcium acetate, Ca(OAc)₂, solution. In this case, such preparation entraps Ca²⁺ ions and (C₂H₃O₂)⁻ ions in the liposome.

The present method next separates extravesicular metal salt solution from the liposome by exposing the liposome to a metal salt-free solution. Such separation creates a gradient of the metal ion across the liposome membrane. Such separation further causes the acid form of the counterion to diffuse out of the liposome, which creates a trans-membrane pH gradient in which the intraliposomal pH is higher than the pH of the metal salt-free solution exterior to the liposome. Thus, after separation of the extravesicular metal salt solution, the liposome has a higher intraliposomal metal ion concentration and a higher intraliposomal pH relative to the extravesicular metal salt-free solution.

For example, continuing with the example above, the present method can separate the extravesicular calcium acetate solution by exposing the liposome to a Na₂SO₄ solution. Such separation creates a Ca²⁺ gradient across the liposome membrane. Such separation also causes C₂H₄O₂ to diffuse out of the liposome thereby creating a trans-membrane pH gradient. Thus, the resultant liposome has a higher intraliposomal Ca²⁺ concentration and a higher intraliposomal pH relative to the extravesicular Na₂SO₄ solution.

Thereafter, the present method exposes the liposome to an isoosmolal solution containing a hydrophobic lactone drug and having a pH lower than the intraliposomal pH. The combination of pH gradient and trans-membrane hydrophobic lactone drug concentration gradient drives the accumulation of the hydrophobic lactone drug into the liposome predominantly in its ring-opened form. Surprisingly, the present method can provide increased loading, increased retention, and prolonged release of the hydrophobic lactone drug.

For example, continuing with the example above, the present method can expose the liposome to an isoosmolal Na₂SO₄ solution containing DB-67 and having a pH lower than the intraliposomal pH. In this case, the combination of pH gradient and trans-membrane DB-67 concentration gradient is the driving force for accumulation of DB-67 into the liposome predominantly in its ring-opened form.

In one embodiment, the present method exposes the liposome with high intraliposomal pH to the isoosmolal solution containing the hydrophobic lactone drug until the pH gradient between the liposome and the isoosmolal solution is dissipated.

As discussed above, the method for active loading of a hydrophobic lactone drug into a liposome disclosed herein can provide increased loading of the hydrophobic lactone drug in the liposome. In one embodiment, the concentration of the hydrophobic lactone drug accumulated in the liposome is at least 5 times higher than concentration achieved by passive loading. In another embodiment, the concentration of the hydrophobic lactone drug accumulated in the liposome is at least 10 times higher than concentration achieved by passive loading. In yet another embodiment, the concentration of the hydrophobic lactone drug accumulated in the liposome is at least 20 times higher than concentration achieved by passive loading.

The present method exploits the pH dependent reversible hydrolysis of the lactone moiety of the hydrophobic lactone drug to improve drug loading, retention, and release. Camptothecins are an exemplary class of hydrophobic lactone drug that can be used in the present method. Any camptothecin can be used. As discussed above, camptothecins are a prominent class of chemotherapeutic agents. Accordingly, when the present method uses camptothecins including DB-67, the resultant liposomes can be used to treat cancer, including malignant solid tumors.

Statins can be used in the present method and are another exemplary class of hydrophobic lactone drug. Exemplary statins include, but are not limited to, atorvastatin, cerivastatin, fluvastatin, lovastatin, mevastatin, pitavastatin, pravastatin, rosuvastatin, and simvastatin.

Statins are a prominent class of drugs that lower cholesterol in subjects with, or susceptible to, cardiovascular disease. Accordingly, when the present method incorporates statins, the resultant liposomes can be used to treat high cholesterol and/or treat cardiovascular disease.

Statins may also have utility in the treatment of cancer. See, for example, K. Hindler, C. S. Cleeland, E. Rivera, and C. D. Collard, The Role of Statins in Cancer Therapy, The Oncologist, Vol. 11, No. 3, 306-315, March 2006. Accordingly, when the present method incorporates statins, the resultant liposomes may be used to treat cancer.

In one embodiment, the hydrophobic lactone drug is poorly soluble in water. In particular, in this embodiment, the hydrophobic lactone drug has an intrinsic solubility (i.e. the solubility of the lactone ring-closed form) of less than 1 mg/ml. Exemplary hydrophobic lactone drugs having such intrinsic solubility include, but are not limited to, camptothecins, statins, and parthenolides.

In another embodiment, the total dissolved solute concentration (including the hydrophobic lactone drug, metal ion, and any buffer) of the aqueous compartment in the liposome is not greater than 0.4 M. Any drug or excipients bound to the membrane or in undissolved solid form (such as entrapped precipitates and salts) are not considered toward the total dissolved solute concentration.

### Preparing a Liposome in the Presence of a Metal Salt Solution

As indicated above, the method for active loading of a hydrophobic lactone drug into a liposome disclosed herein involves preparing a liposome in the presence of a metal salt solution. Prior to making liposomes, the solution of the metal salt is prepared.

The metal salt solution contains a metal ion and a counterion of the metal salt. Preferred metal ions include alkaline earth metal ions and transition metal ions. Exemplary alkaline earth metal ions include, but are not limited to, calcium ions and magnesium ions. Exemplary transition metal ions include, but are not limited to, copper ions, zinc ions, iron ions, and aluminum ions.

The acid form of the counterion of the metal salt can be a highly permeable weak acid. Exemplary highly permeable weak acids include, but are not limited to, monocarboxylic acids (e.g. acetate, formate, etc.) Accordingly, suitable metal salt solutions include solutions of calcium acetate, magnesium acetate, copper acetate, etc.

In one embodiment, the acid form of the counterion of the metal salt is a monocarboxylic acid. The monocarboxylic acid can be selected from the group consisting of acetate and formate.

In one embodiment, the metal salt is an alkaline earth metal salt or a transition metal salt. The alkaline earth metal can be calcium or magnesium. The transition metal can be selected from the group consisting of copper, iron, zinc, and aluminum.

In one embodiment, the metal salt is selected from the group consisting of calcium acetate, magnesium acetate, and copper acetate.

The metal salt solution may include other constituents such as osmotic enhancers, for example, sodium chloride, sucrose, mannitol, etc.

If desired, the pH of the metal salt solution can be adjusted with NaOH or NH₄OH solution to a pH>7. If the pH is adjusted to a pH>7, then the conditions chosen are typically those that do not result in precipitation of the hydroxide salt of the metal ion.

The concentration of the metal salt solution is typically 0.05-0.3 M. The total osmolality of the final metal salt solution is preferably less than 350 mOsm.

In one embodiment, the hydrophobic lactone drug may be added to the metal salt solution. This embodiment utilizes a combination of active and passive loading. While direct addition of hydrophobic lactone drug to the metal salt solution is not necessary, it may be desirable. Typically, to create salt, the hydrophobic lactone drug should be supersaturated inside the liposome. Since uptake of the hydrophobic lactone drug into the liposome is not rapid during active loading, direct addition of the hydrophobic lactone drug to the metal salt solution can help generate nuclei, help the drug quickly gain supersaturation, and reduce the time required to make the intravesicular salt (i.e. the salt of the metal ion and the lactone ring-opened form of the hydrophobic lactone drug). Accordingly, direct addition of the hydrophobic lactone drug to the metal salt solution can increase uptake of the hydrophobic lactone drug into the liposome during active loading.

The amount of drug added to the metal salt solution should be at or slightly below the saturation level (KSP) of the salt so that there is no precipitation during preparation of the liposome itself. If the hydrophobic lactone drug is added to the metal salt solution, pH adjustment of the metal salt solution with NaOH or NH₄OH solution to a pH>7 is typically desired.

In one embodiment, the hydrophobic lactone drug is directly dissolved in the metal salt solution up to its saturation point. The saturation point is the maximum drug concentration that can be dissolved in the metal salt solution without any precipitation.

Blank liposomes for drug loading can be prepared by the hydration-extrusion procedure, which is well known in the art. In order to prepare blank liposomes, weighed amounts of lipids can be hydrated with the metal salt solution above the lipid transition temperature to prepare multilamellar vesicles. The multilamellar vesicles can then be extruded through a membrane of desired pore size (typically 50-200 nm) to prepare unilamellar vesicles. Alternatively, unilamellar vesicles can be prepared by sonication.

Accordingly, in one embodiment, the liposome is made of unilamellar vesicles. Unilamellar vesicles constitute only a single lipid bilayer as opposed to multilamellar vesicles that constitute multiple lipid bilayers.

Preferably, the final liposome suspension comprises a mixture of phospholipids and may also include cholesterol.

A first phospholipid that can be used includes distearoylphosphatidyl choline (DSPC), dipalmitoylphosphatidyl choline (DPPC), diarachidonoyl phosphatidyl choline (DAPC), hydrogenated soy phosphatidyl choline (HSPC), dimyristoylphosphatidyl glycerol (DMPG), dioleoylphosphatidylglycerol (DOPG), dimyristoylphosphatidylcholine (DMPC), phosphatidyl choline (PC), and phosphatidyl ethanolamine (PE). This first phospholipid is usually the major component in the mixture of phospholipids and generally constitutes 70-95% of the mixture.

A second phospholipid that can be used is a pegylated phospholipid. This second phospholipid is typically about 5-10% of the mixture. Pegylation refers to the attachment of a polyethylene glycol segment to the phospholipid. This second phospholipid is usually pegylated phosphatidyl ethanolamine (PE) of any chain length. The pegylated phospholipid can be added either during preparation of unilamellar vesicles or added after preparation of unilamellar vesicles.

Both the first and second phospholipids preferably have the same chain length. For example, DSPC can be used with pegylated DSPE.

Another constituent in the liposomes can be cholesterol.

Following their preparation, liposomes may be allowed to cool to room temperature for several hours and stored in a 5° C. refrigerator until drug loading.

### Separating Extravesicular Metal Salt Solution

As discussed above, the present method separates extravesicular metal salt solution from the liposome by exposing the liposome to a metal salt-free solution. For example, blank liposomes may be allowed to equilibrate, for example at 37° C., and may be dialyzed against a metal-free salt solution to remove extravesicular metal salt solution.

The metal-free salt solution is preferably a solution of NaCl or sucrose or other inorganic solution containing solutes that are membrane impermeable. In one embodiment, the metal salt-free solution has a pH of 7.4 or less.

### Exposing the Liposome to an Isoosmolal Drug Solution

As discussed above, the present method further exposes the liposome to an isoosmolal drug solution. For example, following the separation of extravesicular metal salt solution, blank liposomes can be dialyzed against an isoosmolal solution containing the hydrophobic lactone drug.

The isoosmolal drug solution can be prepared by adding a weighed amount of solid hydrophobic lactone drug to an isoosmolal solution of NaCl or sucrose or other inorganic solutions containing solutes that are membrane impermeable. Alternatively, the isoosmolal drug solution can be prepared by diluting a concentrated solution of hydrophobic lactone drug into an isoosmolal solution of NaCl or sucrose or other inorganic solutions containing solutes that are membrane impermeable.

The pH of the isoosmolal drug solution is any pH at which the hydrophobic lactone drug exists predominantly in its lactone ring-closed form. In general, hydrophobic lactone drugs typically exist predominantly in their lactone ring-closed form at pH<5. However, DB-67 can exists predominantly in its lactone ring-closed form at pH<7.

The pH of the isoosmolal drug solution can be maintained by use of an impermeable buffer such as borate or phosphate as one of the constituents of the isoosmolal drug solution. In one embodiment, the isoosmolal solution containing the hydrophobic lactone drug comprises sodium chloride, citrate buffer, or phosphate buffer.

The temperature during the exposure of the liposome to the isoosmolal drug solution may vary. The temperature for this exposure step is preferably greater than 20° C. If a faster rate of drug loading is desired, the temperature can be as high as 60° C., but preferably not greater than 70° C.

The time of exposure of the liposome to the isoosmolal drug solution may also vary. The time of exposure depends upon the temperature and the membrane permeability of the hydrophobic lactone drug. Accordingly, drug loading can take anywhere from a few minutes to as long as a few days. For example, in the case of DB-67, the exposure time is generally up to 3 days at 40° C.

After the exposure step, liposomes can be allowed to cool to room temperature for several hours and the unentrapped hydrophobic lactone drug can be immediately removed form liposome entrapped hydrophobic lactone drug. Following removal of the unentrapped hydrophobic lactone drug, liposomes are typically stored at a temperature less than 10° C. until use. However, if separation of unentrapped hydrophobic lactone drug is desirable immediately prior to use, then liposomes can be stored without separation of the unentrapped hydrophobic lactone drug.

Separation of unentrapped hydrophobic lactone drug can be achieved using any of the currently well known methods. These separation methods include gel filtration, ultrafiltration, centrifugation, or equilibrium dialysis. In any of these procedures, the unentrapped drug solution outside the liposomes can be exchanged for an isoosmolal solution of NaCl or sucrose or physiologically compatible buffers such as phosphate buffered saline (PBS). The unentrapped drug solution can also be exchanged for a solution of predominantly water, for example, water or a solution of water and alcohol. Following separation, liposomes can be either used immediately or stored at a temperature less than 10° C. until use.

### Liposomal Formulation

Also disclosed herein is a liposomal formulation comprising a liposome and a liquid carrier. In the liposomal formulation, the liposome comprises a hydrophobic lactone drug and has an intraliposomal metal ion concentration higher than the metal ion concentration of the liquid carrier.

The liquid carrier can be an aqueous solution or buffer. The liquid carrier is usually the solution that was used to separate unentrapped hydrophobic lactone drug. For example, the liquid carrier can be a solution of NaCl or sucrose or physiologically compatible buffers such as phosphate buffered saline (PBS). Preferably the liquid carrier is a solution of predominantly water, for example, water or a solution of water and alcohol. The aqueous solution or buffer may be isosmotic, meaning that the osmotic pressure of the aqueous solution or buffer equals the osmotic pressure of the interior of the liposome, so that the liposome does not shrink, expand, or burst. The liquid carrier may include agents to adjust tonicity.

The liposomal formulation can be made according to the method for active loading of a hydrophobic lactone drug into a liposome disclosed herein. When the liposomal formulation is made according to the method disclosed herein, the method includes separating the hydrophobic lactone drug in the liposome from unentrapped hydrophobic lactone drug by exchanging unentrapped drug solution outside the liposomes for the liquid carrier. As discussed above, such separation can be achieved using any of the currently well known methods including gel filtration, ultrafiltration, centrifugation, or equilibrium dialysis.

Since the liposomal formulation can be made according to the method for active loading of a hydrophobic lactone drug into a liposome disclosed herein, the discussion above regarding the properties of the liposome also applies to the liposomes in the liposomal formulation. For example, the details regarding metal ions, hydrophobic lactone drugs, phospholipids, unilamellar vesicles, intrinsic solubility, total dissolved solute concentration, etc. also applies to the liposomes in the liposomal formulation. Similarly, any properties of the liposomes in the liposomal formulation also apply to the liposomes prepared by the active loading method disclosed herein.

The intraliposomal concentration of the hydrophobic lactone drug may vary. In one embodiment, the intraliposomal concentration of the hydrophobic lactone drug is at least 1 mM. In another embodiment, the intraliposomal concentration of the hydrophobic lactone drug is at least 2 mM. In a further embodiment, the intraliposomal concentration of the hydrophobic lactone drug is at least 3 mM.

In yet another embodiment, the osmolality of the liposome formulation is not greater than 0.4 Osm.

### Methods of Treatment and Administration

The liposomal formulation is useful for treating the same diseases or conditions that the hydrophobic lactone drug contained within the liposome is useful for treating. For example, when the hydrophobic lactone drug is a statin, the liposomal formulation can be used to treat high blood cholesterol and/or cardiovascular disease. When the hydrophobic lactone drug is a camptothecin or a statin, the liposomal formulation can be used to treat cancer.

Accordingly, also disclosed herein are methods for treating diseases and conditions comprising administering to a patient in need thereof a pharmaceutically-effective amount of the liposomal formulation. When the hydrophobic lactone drug is a statin, a method of lowering blood cholesterol comprises administering to a patient in need thereof a cholesterol-lowering effective amount of the liposomal formulation. When the hydrophobic lactone drug is a camptothecin, a method of treating cancer comprises administering to a patient in need thereof a cancer-treating effective amount of the liposomal formulation. When the hydrophobic lactone drug is a statin, a method of treating cancer comprises administering to a patient in need thereof a cancer-treating effective amount of the liposomal formulation.

The method of administration may deliver pharmaceutically effective amounts of the hydrophobic lactone drug to a target tissue or to the bloodstream.

To deliver pharmaceutically effective amounts of the hydrophobic lactone drug to a target tissue, the liposomes should generally exhibit several characteristics. Namely, the concentration of the drug encapsulated in the liposomes should be sufficient to enable administration of pharmaceutically effective doses in vivo. After administration, the drug should be sufficiently retained while the liposomes are circulating in the bloodstream to prevent its leakage from the liposomes before the liposomes have collected in the target tissue (e.g. tumor). Once the liposomes have collected in the target tissue, the rate of drug release should not be so slow that the tissue levels of the drug fail to reach adequate concentrations for effective treatment (e.g. effective tumor cell killing/growth inhibition). The active form of the drug should be delivered to the target tissue (e.g. tumor).

The patient or subject in need can be any mammalian species including, but not limited to, human, monkey, cow, sheep, pig, goat, horse, mouse, rat, dog, cat, rabbit, guinea pig, hamster, and horse. Preferably the patient or subject in need is human.

The liposomal formulation can be delivered directly or in pharmaceutical compositions along with suitable carriers or excipients, as is well known in the art. For example, a pharmaceutical composition may include a conventional additive, such as a stabilizer, buffer, salt, preservative, filler and the like, as known to those skilled in the art. Exemplary buffers include phosphates, carbonates, citrates, and the like. Exemplary preservatives include EDTA, EGTA, BHA, BHT and the like.

A pharmaceutically effective amount of the drug can readily be determined by routine experimentation, as can the most effective and convenient route of administration and the most appropriate formulation. Various formulations and drug delivery systems are available in the art. See, e.g., Gennaro, A. R., ed. (1995) Remington's Pharmaceutical Sciences.

Suitable routes of administration may, for example, include oral, topical, transdermal, local by inhalation, rectal, transmucosal, nasal, or intestinal administration and parenteral delivery, including intramuscular, subcutaneous, intramedullary injections, as well as intrathecal, direct intraventricular, intravenous, intraperitoneal, intranasal, or intraocular injections. In addition, the formulations may be administered sublingually or via an aerosol or spray, including a sublingual tablet or a sublingual spray. The formulations may be administered in a local rather than a systemic manner. For example, a suitable formulation can be delivered via injection or in a targeted drug delivery system, such as a depot or sustained release formulation. Other uses, depending on the particular properties of the preparation, may be envisioned by those skilled in the art.

The mode of administration of the liposomal formulations and pharmaceutical formulations thereof may determine the sites and cells in the subject to which the hydrophobic lactone drug will be delivered. The liposomal formulations of the present invention can be administered alone but will generally be administered in admixture with a pharmaceutical carrier selected with regard to the intended route of administration and standard pharmaceutical practice.

As discussed above, the preparations may be injected parenterally, for example, intravenously. Preferably, the route of administration is intravenous. For parenteral administration, they can be used, for example, in the form of a sterile aqueous solution which may contain other solutes, for example, enough salts or glucose to make the solution isotonic. They may also be employed for peritoneal lavage or intrathecal administration via injection. They may also be administered subcutaneously.

For the oral mode of administration, the liposomal formulations and pharmaceutical formulations thereof can be used in the form of tablets, capsules, losenges, troches, powders, syrups, elixirs, aqueous solutions and suspensions, and the like. In the case of tablets, carriers which can be used include lactose, sodium citrate and salts of phosphoric acid. Various disintegrants such as starch, and lubricating agents, such as magnesium stearate, sodium lauryl sulfate and talc, are commonly used in tablets. For oral administration in capsule form, useful diluents are lactose and high molecular weight polyethylene glycols. When aqueous suspensions are required for oral use, the active ingredient is combined with emulsifying and suspending agents. If desired, certain sweetening and/or flavoring agents can be added.

For the topical mode of administration, the liposomal formulations and pharmaceutical formulations thereof may be incorporated into dosage forms such as gels, oils, emulsions, and the like. Such preparations may be administered by direct application as a cream, paste, ointment, gel, lotion or the like.

The pharmaceutical formulations may be manufactured by any of the methods well known in the art, such as by conventional mixing, dissolving granulating, dragee-making, levigating, emulsifying, encapsulating, entrapping, or lyphophilizing processes. As noted above, the formulations can include one or more physiologically acceptable carriers such as excipients and auxiliaries that facilitate processing of active molecules into preparations for pharmaceutical use.

Proper formulation is dependent upon the route of administration chosen. For injection, for example, the composition may be formulated in aqueous solutions, preferably in physiologically compatible buffers such as Hanks' solution, Ringer's solution, or physiological saline buffer. For transmucosal or nasal administration, penetrants appropriate to the barrier to be permeated are used in the formulation. Such penetrants are generally known in the art.

Compositions formulated for parenteral administration by injection, e.g., by bolus injection or continuous infusion can be presented in unit dosage form, e.g., in ampoules or in multi-dose containers, with an added preservative.

For any composition used in the present methods of administration, a pharmaceutically effective dose can be estimated initially using a variety of techniques well known in the art. For example, in a cell culture assay, a dose can be formulated in animal models to achieve a circulating concentration range that includes the IC₅₀ as determined in cell culture. Dosage ranges appropriate for human subjects can be determined, for example, using data obtained from cell culture assays and other animal studies.

A pharmaceutically effective dose of an agent refers to that amount of the agent that results in amelioration of symptoms or a prolongation of survival in a subject. Toxicity and therapeutic efficacy of such molecules can be determined by standard pharmaceutical procedures in cell culture or experimental animals, e.g., by determining the LD₅₀ (the dose lethal to 50% of the population) and the ED₅₀ (the dose pharmaceutically effective in 50% of the population). The dose ratio of toxic to therapeutic effects is the therapeutic index, which can be expressed as the ratio LD₅₀/ED₅₀. Agents that exhibit high therapeutic indices are preferred.

Dosages preferably fall within a range of circulating concentrations that includes the ED₅₀ with little or no toxicity. Dosages may vary within this range depending upon the dosage form employed and the route of administration utilized. The exact formulation, route of administration, and dosage should be chosen, according to methods known in the art, in view of the specifics of a subject's condition.

The amount of liposomal formulation administered will, or course, be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack. The pack or dispenser device may be accompanied by instructions for administration. Compositions comprising a composition of the invention formulated in a compatible pharmaceutical carrier may also be prepared, placed in an appropriate container, and labeled for treatment of an indicated condition.

These and other embodiments of the present invention will readily occur to those of ordinary skill in the art in view of the disclosure herein, and are specifically contemplated.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description. Such modifications fall within the scope of the appended claims.

### Example 1

### Active Loading

A 0.25 M calcium acetate solution was prepared in deionized water and filtered through a 0.2 μm nylon filter. This solution was used to hydrate phospholipids (DSPC: m-PEG DSPE 95:5 mol %) at 60° C. to form a multilamellar vesicle suspension. The multilamellar vesicles were extruded through a 200 nm polycarbonate membrane at 60° C. to obtain a unilamellar vesicle suspension. The unilamellar vesicles were allowed to cool to room temperature for 6 hours and stored at 5° C. until the drug loading procedure. Prior to drug loading, the unilamellar vesicles were dialyzed against 1 L of 0.375 M NaCl solution at 37° C. for 3 hours to remove the extravesicular calcium acetate solution. Following separation of the extravesicular calcium acetate solution, liposomes were placed in a dialysis tube and dialyzed for 48 hours at 40° C. against 500 ml of 0.125 M NaCl solution containing excess DB-67 solid. The pH of the NaCl solution was adjusted every 12 hours to a pH of 7.4 using 1 M NaOH or HCl. After 48 hours liposomes were removed from the dialysis tube and allowed to cool to room temperature for 3 hours. 20 μl of liposome suspension was then applied to a gel filtration column followed by 5 ml of “carbonated” phosphate buffered saline (C-PBS) and 5 ml of the eluent liposome suspension was collected. The liposomes collected from gel filtration were immediately transferred into a dialysis tube and dialyzed at 37° C. against 1 liter of “carbonated” PBS. “Carbonated” PBS is prepared by supplementing phosphate buffered saline (PBS) with a physiological concentration of carbonate. 50 μl samples were taken from inside the dialysis tube at various time intervals and diluted into 900 μl of ice-cold solution of methanol and acetonitrile (2:1, v/v) at −25° C. Following dilution, samples were stored at −25° C. until HPLC analysis.

### Example 2

### Combination of Active and Passive Loading

A 0.1 M calcium acetate solution was prepared in deionized water and the osmolality of this solution was adjusted to 0.25 Osm with NaCl. DB-67 was added to this solution from a stock solution (0.1 M DB-67 in 1 M NaOH) to obtain a drug concentration of 0.5 mM. The pH of the solution was adjusted to a pH of 9 with 1 M NH₄OH. Following pH adjustment, this solution was filtered through a 0.2 μm nylon filter. The filtered solution was used to hydrate phospholipids (DSPC: m-PEG DSPE 95:5 mol %) at 60° C. to form a multilamellar vesicle suspension. The multilamellar vesicles were extruded through a 200 nm polycarbonate membrane at 60° C. to obtain a unilamellar vesicle suspension. The unilamellar vesicles were allowed to cool to room temperature for 6 hours and stored at 5° C. until the drug loading procedure. Prior to drug loading, the unilamellar vesicles were dialyzed against 1 L of 0.125 M NaCl solution at 37° C. for 3 hours to remove the extravesicular calcium acetate solution. Following separation of the extravesicular calcium acetate solution, liposomes were placed in a dialysis tube and dialyzed for 48 hours at 40° C. against 500 ml of 0.125 M NaCl solution containing excess DB-67 solid. The pH of the NaCl solution was adjusted every 12 hours to a pH of 7.4 using 1 M NaOH or HCl. After 48 hours liposomes were removed from the dialysis tube and allowed to cool to room temperature for 3 hours. 20 μl of liposome suspension was then applied to a gel filtration column followed by 5 ml of “carbonated” phosphate buffered saline (C-PBS) and 5 ml of the eluent liposome suspension was collected. The liposomes collected from gel filtration were immediately transferred into a dialysis tube and dialyzed at 37° C. against 1 liter of “carbonated” PBS. 50 μl samples were taken from inside the dialysis tube at various time intervals and diluted into 900 μl of ice-cold solution of methanol and acetonitrile (2:1, v/v) at −25° C. Following dilution, samples were stored at −25° C. until HPLC analysis.

### Example 3

### Active Loading/Combination of Active and Passive Loading

The unilamellar vesicles are prepared as in Examples 1 and 2, but the salt used is magnesium acetate.

### Example 4

### Active Loading/Combination of Active and Passive Loading

The unilamellar vesicles are prepared as in Examples 1 and 2, but the salt used is copper acetate.

### Example 5

### Active Loading/Combination of Active and Passive Loading

The unilamellar vesicles are prepared as in Examples 1 through 4, but the extravesicular solution during drug loading is an isoosmolar citrate buffer (pH=7) instead of the NaCl solution.

### Example 6

### Active Loading/Combination of Active and Passive Loading

The unilamellar vesicles are prepared as in Examples 1 through 4, but the extravesicular solution used during drug loading is an isoosmolar pH 7.4 phosphate buffer.

### Example 7

### Active Loading/Combination of Active and Passive Loading

The unilamellar vesicles are prepared as in Examples 1 through 6, but the chosen hydrophobic lactone drug is SN-38.

### Example 8

### Active Loading/Combination of Active and Passive Loading

The unilamellar vesicles are formed as in Examples 1 through 6, but the chosen hydrophobic lactone drug is karenitecin.

### Example 9

### Active Loading/Combination of Active and Passive Loading

The unilamellar vesicles are formed as in Examples 1 through 6, but the chosen hydrophobic lactone drug is lovastatin.

### Example 10

### Active Loading/Combination of Active and Passive Loading

The vesicles are prepared with any of the buffers or methods of liposome preparation or choice of drug candidate used in Examples 1 through 9, but the separation of unentrapped drug from entrapped drug is achieved by equilibrium dialysis.

### Example 11

### Active Loading/Combination of Active and Passive Loading

The vesicles are prepared with any of the buffers, methods of liposome preparation or choice of drug candidate used in Examples 1 through 10, but the phospholipids used are 90% DSPC and 10% m-PEG DSPE.

### Example 12

### Active Loading/Combination of Active and Passive Loading

The vesicles are prepared with any of the buffers, methods of preparation or choice of drug candidate used in Examples 1 through 10, but the phospholipids used are 95% HSPC and 5% pegylated PE.

### Example 13

### Active Loading/Combination of Active and Passive Loading

The vesicles are prepared with any of the buffers, methods of preparation or choice of drug candidate used in Examples 1 through 10, but the phospholipids used are 95% DMPC and 5% pegylated DMPE.

### Example 14

### Drug Loading, Retention, and Prolonged Release

For the purpose of this study, 200 nm unilamellar vesicles were prepared using calcium acetate solution. Following separation of external calcium acetate by size exclusion chromatography, liposomes were dialyzed against a suspension of DB-67 at 40° C. The pH of the extravesicular drug suspension was periodically adjusted to 7.4 with NaOH or HCl. 0.5 mL of liposome suspension was withdrawn from inside the dialysis tube at various times and liposome entrapped drug was separated from unentrapped drug by size exclusion chromatography. The concentration of DB-67 loaded into vesicles was analyzed by HPLC. At the end of the loading procedure, 5 mL of vesicles were dialyzed against 1 L of pH 7.4 carbonated phosphate buffer (C-PBS) to monitor the release of DB-67. At various times, 100 μL liposome suspension was withdrawn from inside the dialysis tube and was diluted into 900 μL of an organic quenching solution (cold (−25° C.) methanol/acetonitrile, 2:1 (v/v)) and the concentration of DB-67 was measured by a HPLC assay.

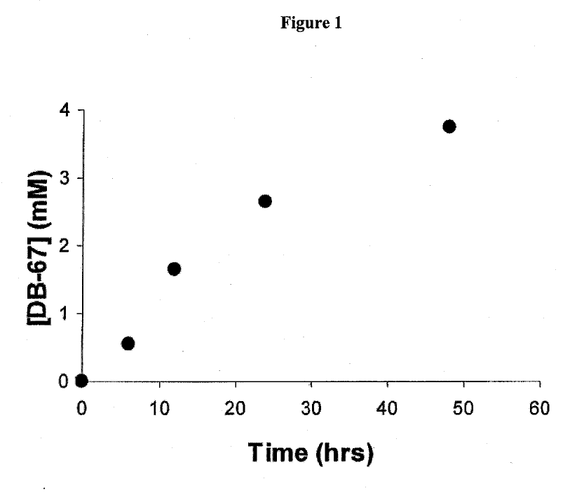

FIG. 1 shows the loading of DB-67 into liposomes after establishing a gradient of pH and calcium ion. More particularly, it shows accumulation of DB-67 into the liposomes as a function of time. The use of this active loading procedure results in the manufacture of liposome suspensions having a suspension drug concentration (all drug being entrapped) as high as 3 mM.

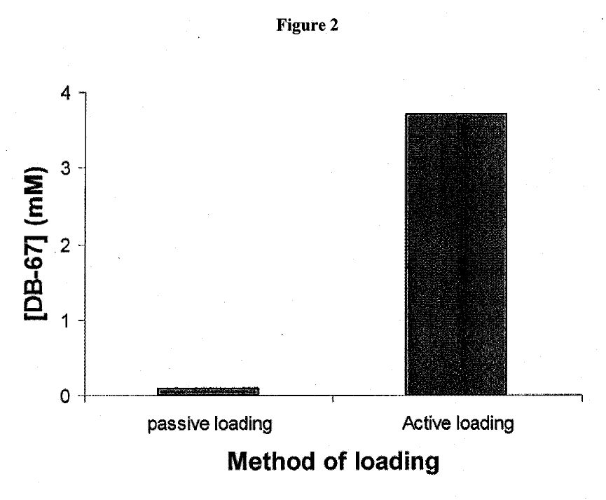

FIG. 2 shows the comparison of the passive liposomal formulation approaches for DB-67 to the current novel approach. Passively loaded liposomes were prepared by the hydration-extrusion procedure. These vesicles were prepared at an intravesicular pH of 4 using 85 mM sodium acetate buffer. Following preparation, liposome entrapped drug was separated from unentrapped drug by size exclusion chromatography and transferred into a dialysis tube and dialyzed against 1 L of pH 7.4 C-PBS buffer. At various times, 100 μL liposome suspension was withdrawn from inside the dialysis tube and was diluted into 900 μL of an organic quenching solution (cold (−25° C.) methanol/acetonitrile, 2:1 (v/v)) and the concentration of DB-67 was measured by a HPLC assay.

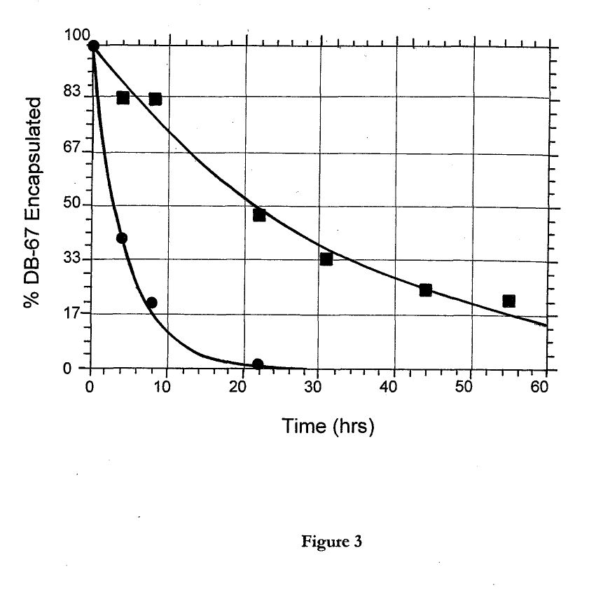

FIG. 3 shows the release of DB-67 from liposomes prepared by using the novel formulation approach. The half-life for release was found to be prolonged to approximately 23 hours compared to 3 hours observed before by using the passive loading approaches for DB-67. This observed half-life translates to retained drug of approximately 50% after 24 hrs with this novel approach. This retention is significantly greater than the previously observed retention of DB-67 or other neutral camptothecins.

Thus, the present method approach allows for increased loading, increased retention, and prolonged release of hydrophobic lactone drugs, which is typically desired when formulating these drugs using liposome technology.

### Example 15

### Drug Loading, Retention, and Prolonged Release

Phospholipids 1,2-distearoyl-sn-glycero-3-phosphatidylcholine (DSPC, >99% purity) and 1,2-distearoyl-sn-glycero-3-phosphoethanolamine-N-[Methoxy(polyethyleneglycol)-2000] (m-PEG DSPE, MW=2806, >99% purity) were purchased as powders from Avanti Polar Lipids (Alabaster, Ala.). DB-67 was obtained from the Novartis Pharmaceuticals Corp. (East Hanover, N.J.). Dialysis tubes (Float-A-Lyzer®, MWCO: 100,000) were purchased from Spectrum Laboratories (Rancho Dominguez, Calif.). Sephadex® G-25 M pre-packed size exclusion columns were obtained from GE Healthcare Bio-sciences Corporation (Piscataway, N.J.). All other reagents and HPLC grade solvents were obtained from Fischer Scientific (Florence, Ky.).

A 0.25 M Ca(OAc)₂ solution was prepared in deionized water and used as the hydration medium to prepare blank vesicles (60 mg DSPC/mL) by the hydration-extrusion procedure at 60° C. discussed in V. Joguparthi, T. X. Xiang, and B. D. Anderson. Liposome transport of hydrophobic drugs: gel phase lipid bilayer permeability and partitioning of the lactone form of a hydrophobic camptothecin, DB-67. J. Pharm. Sci. 97: 400-420 (2008). Two mL of these blank vesicles were immediately transferred into a 60° C. incubator and the outer monolayer was peglyated at 5 mol % by adding a required amount of stock solution (100 mg/mL in deionized water) of m-PEG DSPE. Following the addition of m-PEG DSPE, vesicles were stored at 60° C. for 1 h, subsequently cooled down for 2 h at room temperature and stored at 5° C. until use in drug loading studies.

Blank vesicles (2 mL) were transferred into a dialysis tube and dialyzed at room temperature for 2 h against 1 L of 0.1 M Na₂SO₄ to create a pH gradient across the liposome membrane. Following the generation of pH gradient, vesicles were transferred into a dialysis tube and dialyzed (40° C.) for 2-3 days against 200 mL of 0.1 M Na₂SO₄ containing 0.1 mg/mL DB-67. The dialysate drug suspension was prepared by a pH adjustment method that enables the preparation of an initial supersaturated solution of DB-67. For preparing this solution, 20 mg of DB-67 was added to 200 mL of 0.1 M Na₂SO₄ and pH of the solution was adjusted (with 1 M NaOH) to 10.5 under continuous stirring. After dissolution of the drug, the pH of the solution was dropped to pH 7.5 (with 1 M HCl) and immediately used in drug loading. The pH of the dialysate DB-67 suspension changed during the loading process and was periodically (every ˜12 h) adjusted back to pH 7.5. At various times during the dialysis, 20 μL of liposome suspension was withdrawn from inside the dialysis tube, loaded on a Sephadex® column (pre-equilibrated with 0.1 M Na₂SO₄) and eluted with 5 mL of 0.1 M Na₂SO₄ solution. The eluent suspension from 2-5 mL was collected and 100 μL of this suspension was diluted into 900 μL of an organic quenching solution (cold (−25° C.) methanol/acetonitrile, 2:1 (v/v)) and stored at −25° C. until analysis. At the end of the drug loading process (after ˜50 h), vesicles were stored at 5° C. until use in release studies.

200 μL of liposome suspension was loaded onto a Sephadex® column, eluted with 5 mL of carbonated phosphate buffered saline (C-PBS) as discussed in V. Joguparthi, T. X. Xiang, and B. D. Anderson. Liposome transport of hydrophobic drugs: gel phase lipid bilayer permeability and partitioning of the lactone form of a hydrophobic camptothecin, DB-67. J. Pharm. Sci. 97: 400-420 (2008), and the eluent suspension from 2-5 mL was collected and dialyzed against 1 L of C-PBS buffer. C-PBS buffer was prepared by adding a physiological concentration of carbonate (24 mM) to PBS, with pH and osmolality maintained at physiologically relevant levels (pH 7.4, 298 mOsm). Separate release studies were also conducted by diluting the Sephadex® eluent liposome suspension to a desired lipid concentration and dialyzing the vesicles against 1 L of C-PBS buffer. At various times 100 μL samples were withdrawn from the dialysis tube and diluted into 900 μL of the organic quenching solution (cold (−25° C.) methanol/acetonitrile, 2:1 (v/v)) and stored at −25° C. until analysis.

Samples were analyzed by a previously validated HPLC method with fluorescence detection discussed in V. Joguparthi, T. X. Xiang, and B. D. Anderson. Liposome transport of hydrophobic drugs: gel phase lipid bilayer permeability and partitioning of the lactone form of a hydrophobic camptothecin, DB-67. J. Pharm. Sci. 97: 400-420 (2008). Standards for DB-67 lactone (10-50 nM) and carboxylate (10-100 nM) were prepared in methanol and pH 10.5 carbonate buffer (10 mM) respectively and all samples were diluted into this range in the organic quenching solution.

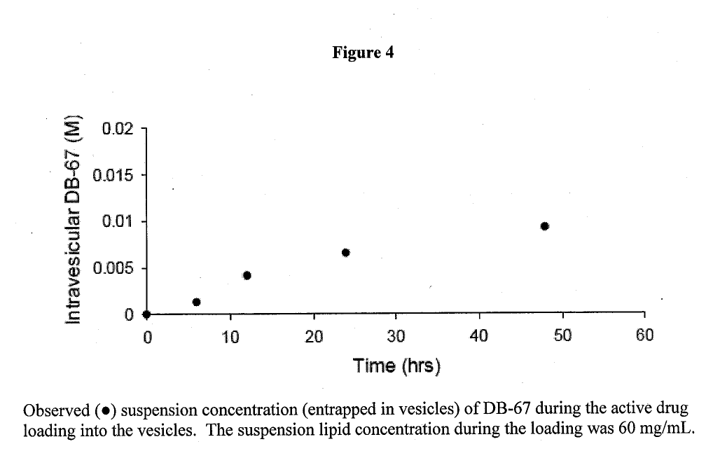

FIG. 4 shows the experimentally observed loaded concentration of DB-67 in these studies. The suspension concentration of DB-67 at the end of the loading was ˜9.2 mM. This corresponds to an intravesicular DB-67 concentration of ˜21 mM (˜10 mg/mL).

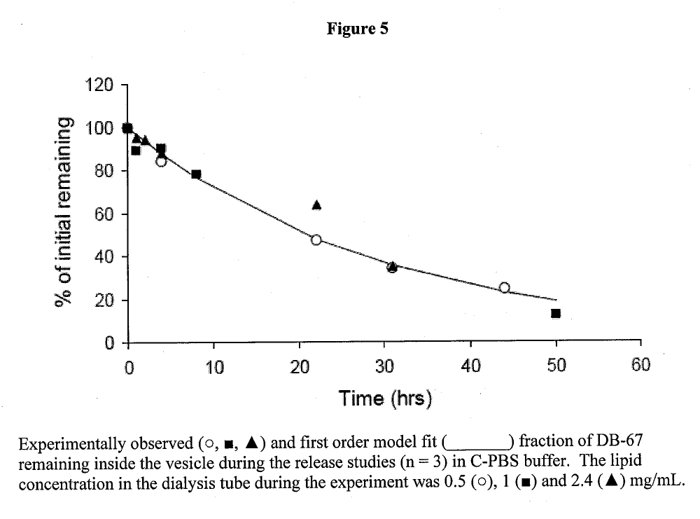

FIG. 5 shows the concentration versus time profile of DB-67 observed during these release studies. The observed concentration versus time profiles were fit to a first order release model to estimate the half-life for liposome retention. The half-life for release was found to be 22±6 h. This half-life was significantly longer than that observed by passive loading methods (˜12 h) in the absence of calcium.

### Example 16

### Animal Pharmacokinetic Studies

Phospholipids 1,2-distearoyl-sn-glycero-3-phosphatidylcholine (DSPC, >99% purity) and 1,2-distearoyl-sn-glycero-3-phosphoethanolamine-N-[methoxy-(polyethyleneglycol)-2000] (m-PEG DSPE, MW=2806, >99% purity) were purchased as powders from Avanti Polar Lipids (Alabaster, Ala.). DB-67 was obtained from the Novartis Pharmaceuticals Corp. (East Hanover, N.J.). Blank plasma used in preparation of calibrators and quality control solutions was obtained from Abacell Corp. (San Mateo, Calif.). Consumables were treated with AquaSil™ siliconizing reagent (Pierce, Rockford, Ill.). Siliconized pipet tips were obtained from Cole-Palmer and amber siliconized microcentrifuge tubes were obtained from Crystalgen Inc. (Plainview, N.Y.). Hydroxypropyl-β-cyclodextrin (HPβCD, degree of substitution=2.94, MW=1305.5) was obtained from American Maize-Products Company (Hammond, Ind.). Heparin (heparin sodium 1000 IU) was obtained from Baxter (Deer Field, Ill.). Dialysis tubes (Float-A-Lyzer®, MWCO: 100,000) and pre-cut dialysis membrane discs (MWCO: 12000-14000) were purchased from Spectrum Laboratories (Rancho Dominguez, Calif.). Sephadex® G-25 M pre-packed size exclusion columns were obtained from GE Healthcare Bio-sciences Corp. (Piscataway, N.J.). All other reagents were purchased from Fischer Scientific (Florence, Ky.) and HPLC grade solvents were obtained from VWR Scientific (Muskegon, Mich.).

A film of the desired lipids, DSPC and m-PEG-DSPE (95:5 mol %) was prepared by dissolving weighed amounts of lipids in chloroform, distributing into glass test tubes at 120 mg of total lipid per tube, evaporating chloroform under N₂ and drying in vacuo at 40° C. overnight. The lipid films were stored at 5° C. until use. Two pegylation procedures were employed in the present studies. Liposomes were pegylated during vesicle preparation or after preparation of vesicles. For liposomes pegylated after unilamellar vesicle preparation, lipid films (120 mg/tube) were prepared using DSPC (100 mol %). These lipid films were employed in preparation of all liposomes employed in these studies.

Blank liposomes were prepared according to the following procedure. Two mL of 85 mM Na acetate buffer solution (pH 4, osmolality adjusted to 300 mOsm with NaCl) was added to a test tube containing 120 mg lipid film and blank unilamellar vesicles (DSPC: m-PEG DSPE 95:5 mol %) were prepared at 60 mg/mL (total lipid concentration) by the hydration-extrustion procedure (explained in V. Joguparthi, T. X. Xiang, and B. D. Anderson. Liposome transport of hydrophobic drugs: gel phase lipid bilayer permeability and partitioning of the lactone form of a hydrophobic camptothecin, DB-67. J. Pharm. Sci. 97: 400-420 (2008)) at 60° C. Following preparation, vesicles were allowed to cool down at room temperature for 2 h and were stored at 5° C. until use in animal injections. On the day of pharmokinetic studies, blank vesicles were warmed up at 37° C. and vesicles were spiked with DB-67 using a stock solution (100 mg/mL) of DB-67 in DMSO to obtain a 1 mg/mL liposome suspension of DB-67. The spiked vesicles were immediately injected into animals at the desired dose (see dosing section).

Liposomes were prepared with high intraliposomal pH according to the following passive loading procedure. A 20 mM DB-67 solution was prepared in 85 mM Na carbonate buffer (pH 9.5, osmolality adjusted to 298 mOsm with NaCl) and 2 mL of this solution was used to prepare unilamellar vesicles (DSPC: m-PEG DSPE=95:5 mol %) by the hydration-extrusion procedure (explained in V. Joguparthi, T. X. Xiang, and B. D. Anderson. Liposome transport of hydrophobic drugs: gel phase lipid bilayer permeability and partitioning of the lactone form of a hydrophobic camptothecin, DB-67. J. Pharm. Sci. 97: 400-420 (2008)) at 60° C. Following preparation, vesicles ([lipid]=60 mg/mL) were allowed to cool down at room temperature for 2 h and were subsequently stored at 5° C. One day prior to the start of the animal studies, liposomes were dialyzed overnight (37°) against 2 L of 85 mM Na carbonate buffer (same buffer employed in vesicle preparation with pH adjusted to the pH of the vesicles) to remove unentrapped drug from the vesicles. During the animal studies, 1 mL of vesicles were loaded onto a Sephadex® size exclusion column (pre-equilibrated with 50 mL of pH 9.5 carbonate buffer used to prepare vesicles), eluted by 5 mL of the carbonate buffer (same buffer employed in vesicle preparation with pH adjusted to pH of the vesicles) and the eluent liposome fraction from 2.5-4.5 mL was collected and injected into mice at the desired dose (see dosing section).

Two mL of 0.25 M Ca(OAc)₂ solution was used to prepare unilamellar DSPC vesicles ([lipid]=60 mg/mL) by the hydration-extrusion procedure. Following preparation, vesicles were pegylated at 60° C. (using m-PEG DSPE) on the outer monolayer as described in V. Joguparthi and B. D. Anderson. Effect of Cyclodextrin Complexation on the Liposome Permeability of a Model Hydrophobic Weak Acid. Pharmaceutical Research 25 (11):2505-2515 (2008) and Uster, P. S., et al., Insertion of poly(ethylene glycol) derivatized phospholipid into pre-formed liposomes results in prolonged in vivo circulation time. FEBS Lett. 386:243-6 (1996). Following peglyation, vesicles (2 mL) were allowed to cool down for 2 h at room temperature and were dialyzed against 0.1 M Na₂SO₄ solution for 3 h to remove unentrapped Ca (OAc)₂ and generate a trans-bilayer pH gradient. After the removal of extravesicular Ca(OAc)₂, vesicles were dialyzed for 3 days against 200 mL of a 0.1 mg/mL suspension of DB-67 (40° C.) in 0.1 M Na₂SO₄ to actively load DB-67 into the vesicles. Due to the low aqueous solubility of DB-67, the dialysate DB-67 suspension was prepared by a pH adjustment method discussed in Xiang, T. X., Anderson, B. D., Stable supersaturated aqueous solutions of silatecan 7-t-butyldimethylsilyl-10-hydroxycamptothecin via chemical conversion in the presence of a chemically modified beta-cyclodextrin. Pharm Res. 19, 1215-22 (2002). To prepare the dialysate suspension of DB-67, weighed (200 mg) amount of DB-67 was added to 0.1 M Na₂SO₄ and the pH of this solution was adjusted to 10.5 using 1 M NaOH. This pH adjustment enabled the solubilization of the DB-67 powder. Following the solubilization, the pH of the drug solution was slowly adjusted back to a pH of 7.5 using 1 M HCl. During the drug loading, the dialysate drug suspension was continually stirred to avoid the sedimentation of any drug precipitate. The pH of the extravesicular dialysate was observed to change during drug loading and this pH was periodically (every 12 h) adjusted back to pH 7.5. At the end of the drug loading period (after ˜72 h), vesicles were removed from the dialysis tube and stored in a 5° C. refrigerator until animal studies. On the day of the animal studies, the extravesicular drug was removed by size exclusion on Sephadex® columns as described earlier prior to injection into mice.

On the day of the animal studies, the entrapped concentration of DB-67 was analyzed 30 min before injection into mice.

DB-67 lactone and carboxylate plasma concentrations were analyzed by an isocratic HPLC method with fluorescence detection as described in Horn, J., et al. Validation of an HPLC method for analysis of DB-67 and its water soluble prodrug in mouse plasma. J Chromatogr B Analyt Technol Biomed Life Sci. 844, 15-22 (2006). Separation was achieved on a reverse phase C-18 column (Waters Nova-Pak, 4 μm, 3.9×150 mm) and mobile phase consisted of a mixture of 0.15 M NH₄OAc (containing 10 mM tetrabutylammonium dihydrogenphosphate (pH 6.5)) and acetonitrile (65:35, (v/v)). DB-67 lactone (1-300 ng/mL) and carboylate (2.5-300 ng/mL) standards were prepared in mobile phase and all samples from extraction were diluted in mobile phase as required prior to analysis. The lipid concentration of the liposome suspension dosed into animals was analyzed by HPLC method with evaporative light scattering detection (ELSD).

Due to differences in drug loading by various formulation procedures, it was not possible to precisely control the dose of drug injected into animals. Instead, the suspension lipid concentration (˜30 mg/mL) of all the formulations employed in these studies was controlled prior to animal injection. The target drug dose in these studies was 10 mg/kg. The injection volume was ˜140-150 μL per animal. The weight of the animals employed in these studies was close to each other (21-24 gm) and an average weight of 23 gm was used to estimate dose. The final drug dose administered into each animal was calculated based on the average injected volume, average animal weight, and the formulation concentration of DB-67.

Table 2 shows the final drug and lipid dose for each formulation administered into animals. The dose of DB-67 was different between the various formulations but within an order of magnitude. Therefore, the small differences in the DB-67 dose were assumed to not affect the pharmokinetics of the liposomal DB-67.

TABLE 2
Dose of liposome formulations employed
in the pharmacokinetic studies in mice

| | Lipid | DB-67 |
|---|---|---|
| Method of Loading | (mg/kg) | (mg/kg) |
| Blank vesicles spiked with DB-67 lactone | 190.7 | 6.2 |
| High intravesicular pH (pre-pegylation) | 186 | 4.4 |
| Active loading in the presence of Ca²⁺ | 179.1 | 5.5 |

The pH and osmolality (freezing point depression method (Model 110 Osmometer, Fiske Associates, Norwood, Mass.)) of all the liposome formulations was monitored during each step of the formulation process. The pH and osmolality of the buffers and dialysate solutions employed used in various steps all adjusted to the pH (with 0.1 N HCl or NaOH) and osmolality (with NaCl) of the liposome suspension. The particle size of all the liposome suspensions was measured prior to the size exclusion step before animal injections by dynamic light scattering (DLS) using Malvern Zetasize-3000 (Malvern Instruments Ltd, Malvern, UK).

The pH, particle size and osmolality of all formulations were measured prior to the size exclusion performed before injection into animals. Table 3 shows the pH, particle size, and osmolality of the formulations employed in these studies.

TABLE 3
Measured pH, osmolality and particle size of
liposomes employed in pharmacokinetic studies

| | | Particle Size | Osmo- |
|---|---|---|---|
| | Final | (nm, Mean ± | lality |
| Method of Loading | pH | S.D.) | (mOsm) |
| Blank vesicles spiked with DB-67 lactone | 4.1 | 139 ± 44 | 296 |
| High intravesicular pH (pre-pegylation) | 9.43 | 146 ± 40 | 301 |
| Active loading in the presence of Ca²⁺ | 7.30 | 143 ± 49 | 297 |

All animal experiments were approved by the University of Kentucky Institutional Animal Care and Use Committee. Female C57BL/6 mice (Harlan, Indianapolis, Ind.) weighing between 18-24 gm were employed in these experiments. Three animals were used per time point in the pharmacokinetic studies and each animal was sampled at three to four different time points over the course of a week. Liposomal formulations were administered as a bolus into the lateral tail vein. Following administration, blood samples of approximately 75 μL were taken from the saphenous vein at 5, 30 min and 1, 1.5, 3, 6, 12, 24, 36 h and collected in heparinized microcentrifuge tubes. For the lipsomal formulation prepared by the active loading method, samples were taken at additional time points of 57 h and 72 h. The collected blood was immediately centrifuged at 1000 RPM for 5 min to separate plasma. DB-67 was extracted from plasma (by centrifugation at 1000 RPM for 5 min) using methanol stored on dry ice (plasma:methanol 1:4). Following extraction, samples were stored at −80° C. until analysis.

The plasma concentration versus time profiles of all formulations were normalized with respect to administered dose and fit using the nonlinear regression software Scientist® (Micromath Scientific Software, St. Louis, Mo.) to the following biexponential equation: C=C1*e^{-k1t}+C2*e^{-k2t} where C is the total drug concentration in plasma at any given time, C1 and C2 are the concentration coefficients of the first and second phase and k1 and k2 are the rate constants for the first and second phase, respectively.

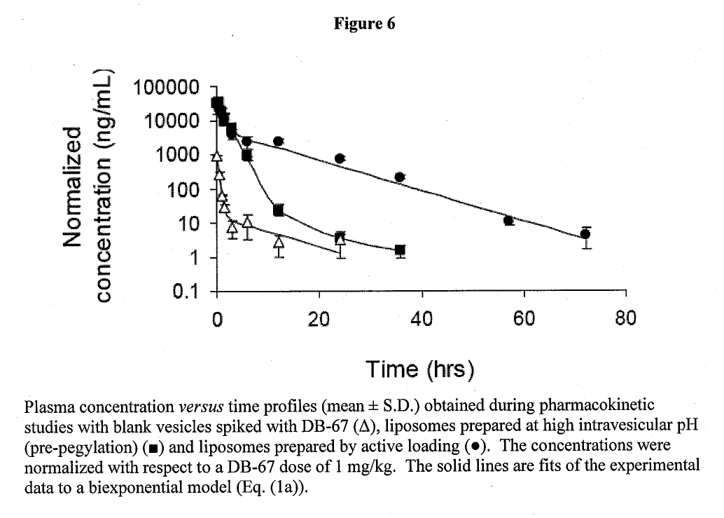

FIG. 6 shows the DB-67 plasma concentration versus time profiles of the blank vesicles spiked with DB-67, liposomes prepared at high intravesicular pH (passive loading), and liposomes prepared by active loading.

### Example 17

### Efficacy of Db-67 in Non-Small Cell Lung Cancer (H460) Xenografts in Mice

Non-small cell lung cancer (H460) tumor was implanted in the flank region of nu/nu mice (body weight 20-25 g). When the tumors were palpable, mice (n=7 per treatment group) were randomized to four treatment groups and received a) control (5% dextrose in water [D5W] intravenously; b) 7.5 mg/kg/day intravenously for 5 days per cycle (1 cycle=21 days); c) 3.75 mg/kg/day intravenously for 10 days per cycle; or d) 2.5 mg/kg/day intravenously for 15 days per cycle. The maximum tolerated dose (MTD) of DB-67 administered by the intravenous route was determined to be 7.5 mg/kg/day for 5 days.

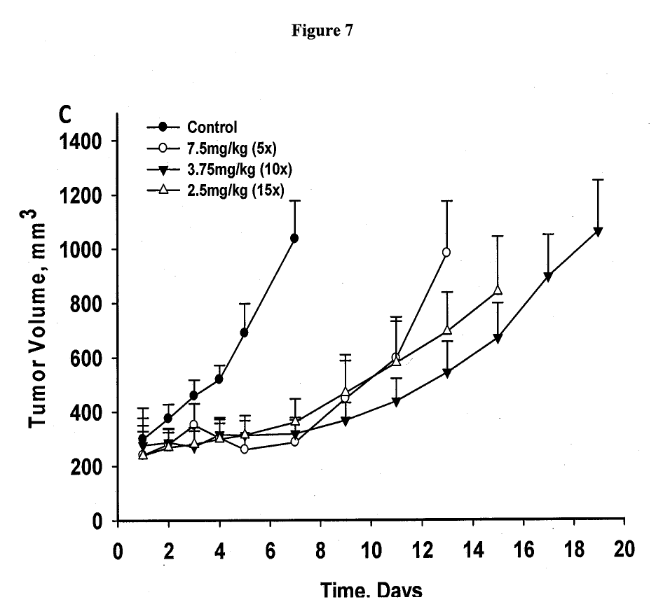

The width and length of the tumors were measured using a caliper every other day for the duration of the study. Tumor volume was calculated using the following formula:

$$V = PI ⋆ \frac{a ⋆ {b}^{2}}{6}$$

where V is tumor volume in mm³, PI=3.1416, a=size of the longest side in millimeters, and b=size of the shortest side in millimeters. Mice were euthanized when their tumor volume reached 1500 mm³ for humane reasons. FIG. 7 shows tumor volume as a function of time for the four treatment groups.

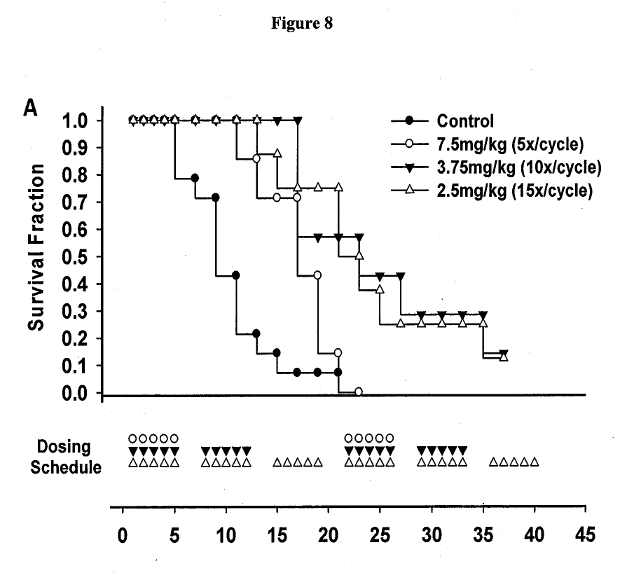

FIG. 8 is a plot showing dosing schedule for the four treatment groups and the survival fraction for the four treatment groups as a function of dosing schedule. Comparison between the median survivals of different treatment groups was done using Kaplan-Meir survival analysis.

FIGS. 7 and 8 demonstrate that protracted dosing of nonliposomal DB-67 is effective in treating non-small cell lung cancer (H460) in mice.

It will be appreciated that, although specific embodiments of the invention have been described herein for purposes of illustration, various modifications may be made without departing from the spirit and scope of the invention. All such modifications and variations are intended to be included herein within the scope of this disclosure and the present invention and protected by the following claims.

That which is claimed is:

1. A method for active loading of a hydrophobic lactone drug into a liposome, the method comprising:
preparing a liposome in the presence of a metal salt solution, an acid form of a counterion of the metal salt being membrane permeable, such that the liposome preparation contains entrapped metal ion;
forming a liposome with high intraliposomal pH by separating extravesicular metal salt solution from the liposome by exposing the liposome to a metal salt-free solution, resulting in diffusion of the acid form of the counterion out of the liposome and formation of an intraliposomal pH higher than that of the metal salt-free solution; and
exposing the liposome with high intraliposomal pH to an isoosmolal solution containing a hydrophobic lactone drug, the isoosmolal solution having a pH lower than the intraliposomal pH, such that the hydrophobic lactone drug accumulates in the liposome predominantly in its ring-opened form.

2. The method of claim 1, wherein the liposome with high intraliposomal pH is exposed to the isoosmolal solution containing the hydrophobic lactone drug until a pH gradient between the liposome and the isoosmolal solution is dissipated.

3. The method of claim 1, wherein the concentration of hydrophobic lactone drug accumulated in the liposome is at least 5 times higher than concentration achieved by passive loading.

4. The method of claim 1, wherein the concentration of hydrophobic lactone drug accumulated in the liposome is at least 10 times higher than concentration achieved by passive loading.

5. The method of claim 1, wherein the concentration of hydrophobic lactone drug accumulated in the liposome is at least 20 times higher than concentration achieved by passive loading.

6. The method of claim 1, wherein the metal salt solution comprises the hydrophobic lactone drug.

7. The method of claim 1, wherein the acid form of the counterion of the metal salt is a monocarboxylic acid.

8. The method of claim 7, wherein the monocarboxylic acid is selected from the group consisting of acetate and formate.

9. The method of claim 1, wherein the metal salt is a alkaline earth metal salt or a transition metal salt.

10. The method of claim 9, wherein the alkaline earth metal is calcium or magnesium.

11. The method of claim 9, wherein the transition metal is selected from the group consisting of copper, iron, zinc and aluminum.

12. The method of claim 1, wherein the metal salt is selected from the group consisting of calcium acetate, magnesium acetate and copper acetate.

13. The method of claim 1, wherein the isoosmolal solution containing the hydrophobic lactone drug comprises sodium chloride, citrate buffer, or phosphate buffer.

14. The method of claim 1, wherein the metal salt-free solution has a pH of 7.4 or less.

15. The method of claim 1, further comprising separating the hydrophobic lactone drug in the liposome from unentrapped hydrophobic lactone drug by gel filtration, equilibrium dialysis, ultrafiltration, or centrifugation.

16. The method of claim 1, wherein the hydrophobic lactone drug is a camptothecin, statin or parthenolide.

17. The method of claim 16, wherein the hydrophobic lactone drug is a camptothecin.

18. The method of claim 17, wherein the camptothecin is selected from the group consisting of DB-67, SN-38, topotecan, irinotecan, 9-nitro-camptothecin, lurtotecan, exatecan, gimatecan and karenitecin.

19. The method of claim 18, wherein the camptothecin is DB-67.

20. The method of claim 16, wherein the hydrophobic lactone drug is a statin.

21. The method of claim 1, wherein the liposome comprises a mixture of phospholipids.

22. The method of claim 21, wherein the liposome further comprises cholesterol.

23. The method of claim 21, wherein the mixture of phospholipids comprises a first phospholipid selected from the group consisting of distearoylphosphatidyl choline, dipalmitoylphosphatidyl choline, diarachidonoyl phosphatidyl choline, hydrogenated soy phosphatidyl choline, dimyristoylphosphatidyl glycerol, dioleoylphosphatidylglycerol, dimyristoylphosphatidylcholine, phosphatidyl choline and phosphatidyl ethanolamine, and a second phospholipid which is a pegylated phospholipid.

24. The method of claim 1, wherein the liposome is made of unilamellar vesicles.

25. The method of claim 1, wherein the hydrophobic lactone drug in a lactone ring-closed form has a solubility in water less than 1 mg/ml.

26. The method of claim 1, wherein the total solute concentration in the aqueous compartment of the liposome is 0.4 M or less.

27. A liposome formed by the method of claim 1.

28. A liposome formed by the method of claim 2.

29. A liposomal formulation comprising a liposome and a liquid carrier, the liposome comprising a hydrophobic lactone drug and having an intraliposomal metal ion concentration higher than the metal ion concentration of the liquid carrier.

30. The liposomal formulation of claim 29, wherein the metal ion is an alkaline earth metal ion or a transition metal ion.

31. The liposomal formulation of claim 30, wherein the alkaline earth metal is calcium or magnesium.

32. The liposomal formulation of claim 30, wherein the transition metal is selected from the group consisting of copper, iron, zinc and aluminum.

33. The liposomal formulation of claim 29, wherein the hydrophobic lactone drug is a camptothecin, statin or parthenolide.

34. The liposomal formulation of claim 33, wherein the hydrophobic lactone drug is a statin.

35. The liposomal formulation of claim 33, wherein the hydrophobic lactone drug is a camptothecin.

36. The liposomal formulation of claim 35, wherein the camptothecin is selected from the group consisting of DB-67, SN-38, topotecan, irinotecan, 9-nitro-camptothecin, lurtotecan, exatecan, gimatecan and karenitecin.

37. The liposomal formulation of claim 36, wherein the camptothecin is DB-67.

38. The liposomal formulation of claim 29, wherein the liposome comprises a mixture of phospholipids.

39. The liposomal formulation of claim 38, wherein the liposome further comprises cholesterol.

40. The liposomal formulation of claim 38, wherein the mixture of phospholipids comprises a first phospholipid selected from the group consisting of distearoylphosphatidyl choline, dipalmitoylphosphatidyl choline, diarachidonoyl phosphatidyl choline, hydrogenated soy phosphatidyl choline, dimyristoylphosphatidyl glycerol, dioleoylphosphatidylglycerol, dimyristoylphosphatidylcholine, phosphatidyl choline and phosphatidyl ethanolamine, and a second phospholipid which is a pegylated phospholipid.

41. The liposomal formulation of claim 29, wherein the liposome is made of unilamellar vesicles.

42. The liposomal formulation of claim 29, wherein the intraliposomal concentration of hydrophobic lactone drug is at least 1 mM.

43. The liposomal formulation of claim 29, wherein the intraliposomal concentration of hydrophobic lactone drug is at least 2 mM.

44. The liposomal formulation of claim 29, wherein the intraliposomal concentration of hydrophobic lactone drug is at least 3 mM.

45. The liposomal formulation of claim 29, wherein the osmolality of the liposomal formulation is not greater than 0.4 Osm.

46. A method of lowering blood cholesterol comprising administering to a patient in need thereof a cholesterol-lowering effective amount of a liposomal formulation of claim 34.

47. A method of treating cancer comprising administering to a patient in need thereof a cancer-treating effective amount of a liposomal formulation of claim 35.

48. A method of treating cancer comprising administering to a patient in need thereof a cancer-treating effective amount of a liposomal formulation of claim 34.
